(19) United States
(12) Patent Application Publication
(10) Pub. No.: US 2018/0098750 A1
(43) Pub. Date: Apr. 12, 2018

(54) ULTRASOUND TRANSDUCER WITH VARIABLE PITCH

(71) Applicant: General Electric Company, Schenectady, NY (US)

(72) Inventors: Bruno Hans Haider, Rehoboth Beach, DE (US); David Martin Mills, Niskayuna, NY (US); Scott William Easterbrook, Bainbridge Island, WA (US); Todor Sheljaskow, Scottsdale, AZ (US)

(21) Appl. No.: 15/287,206

(22) Filed: Oct. 6, 2016

(51) Int. Cl. A61B 8/00; A61B 8/14; A61B 8/08

(52) U.S. Cl. CPC A61B 8/4488; A61B 8/4444; A61B 8/461; A61B 8/145; A61B 8/5207; A61B 8/4494

(57) ABSTRACT

Methods and systems are provided for ultrasound transducers with variable pitch. In one embodiment, an ultrasound probe comprises a plurality of transducer elements arranged in an array, wherein a pitch between adjacent transducer elements increases from a first pitch in a center of the array to a second pitch at an edge of the array, and wherein the pitch gradually varies between the first pitch and second pitch for transducer elements positioned between the center and the edge. In this way, an ultrasound probe can be operated in multiple modes while avoiding image artifacts caused by an abrupt change in pitch.

### FIELD

Embodiments of the subject matter disclosed herein relate to ultrasound imaging, and more particularly, to ultrasound transducers with variable pitch.

### BACKGROUND

Medical diagnostic ultrasound is an imaging modality that employs ultrasound waves to probe the acoustic properties of the body of a patient and produce a corresponding image. Generating of sound wave pulses and detection of returning echoes is typically accomplished by an ultrasound probe having one or more transducers. Such transducers typically include electromechanical elements capable of converting electrical energy into mechanical energy for transmission of ultrasonic waves into patient tissue and mechanical energy back into electrical energy when the reflected ultrasonic waves reach the transducers.

Different transducers may be used for different types of imaging. For example, a linear transducer array may be used for cardiac imaging, while a curvilinear array may be used for abdominal imaging. However, the use of different transducer arrays is cumbersome for mobile ultrasound devices, as a user of a mobile ultrasound device must carry multiple transducer arrays and switch transducer arrays based on the desired imaging application. Thus, a transducer array that may be used for phased array scans as well as curvilinear scans is desirable.

One approach to providing a dual mode ultrasound transducer is described in U.S. Pat. No. 8,551,004 to Adams et al. As described therein, an ultrasound transducer includes transducer elements in a central portion with a finer pitch than the pitch of the transducer elements of the lateral portions. However, the pitch is the same across the transducer elements in the central portion, and the pitch is the same across the transducer elements of the lateral portions. Consequently, the boundary between the first, finer pitch in the central region and the second, coarser pitch in the lateral regions introduces image artifacts.

### BRIEF DESCRIPTION

In one embodiment, an ultrasound probe comprises a plurality of transducer elements arranged in an array, wherein a pitch between adjacent transducer elements increases from a first pitch in a center of the array to a second pitch at an edge of the array, and wherein the pitch gradually varies between the first pitch and second pitch for transducer elements positioned between the center and the edge. In this way, an ultrasound probe can be operated in multiple modes while avoiding image artifacts caused by an abrupt change in pitch.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an ultrasonic imaging system according to an embodiment of the invention;

FIG. 2 shows a diagram illustrating a convex transducer with variable pitch according to an embodiment of the invention;

FIG. 3 shows a diagram illustrating a linear transducer with variable pitch according to an embodiment of the invention;

FIG. 4 shows a graph illustrating an example relation between pitch and transducer element position according to an embodiment of the invention;

FIG. 5 shows a graph illustrating another example relation between pitch and transducer element position according to an embodiment of the invention;

FIG. 6 shows a diagram illustrating a convex transducer with variable pitch and variable sub-dicing according to an embodiment of the invention;

FIG. 7 shows a diagram illustrating a linear transducer with variable pitch and variable sub-dicing according to an embodiment of the invention;

FIG. 8 shows a graph illustrating an example relation between sub-slice depth and pitch according to an embodiment of the invention;

FIG. 9 shows a graph illustrating another example relation between sub-slice depth and pitch according to an embodiment of the invention; and

FIG. 10 shows a high-level flow chart illustrating an example method for performing an ultrasound scan according to an embodiment of the invention.

### DETAILED DESCRIPTION

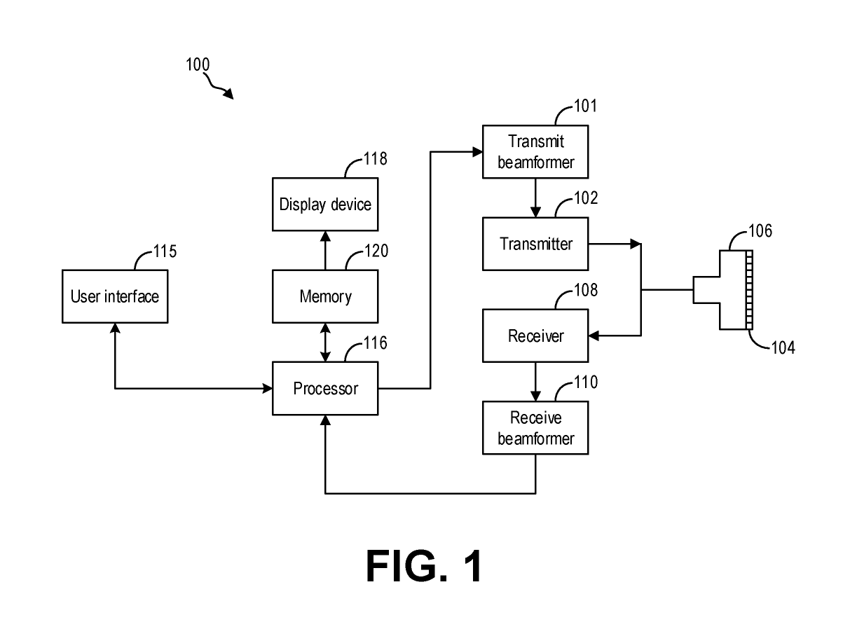

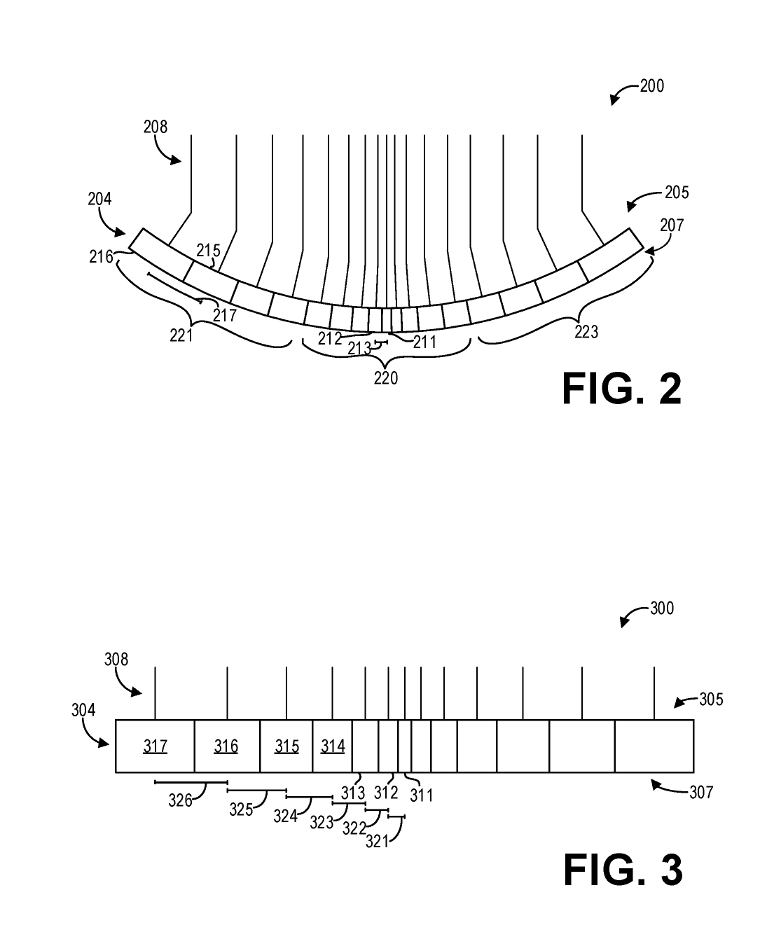

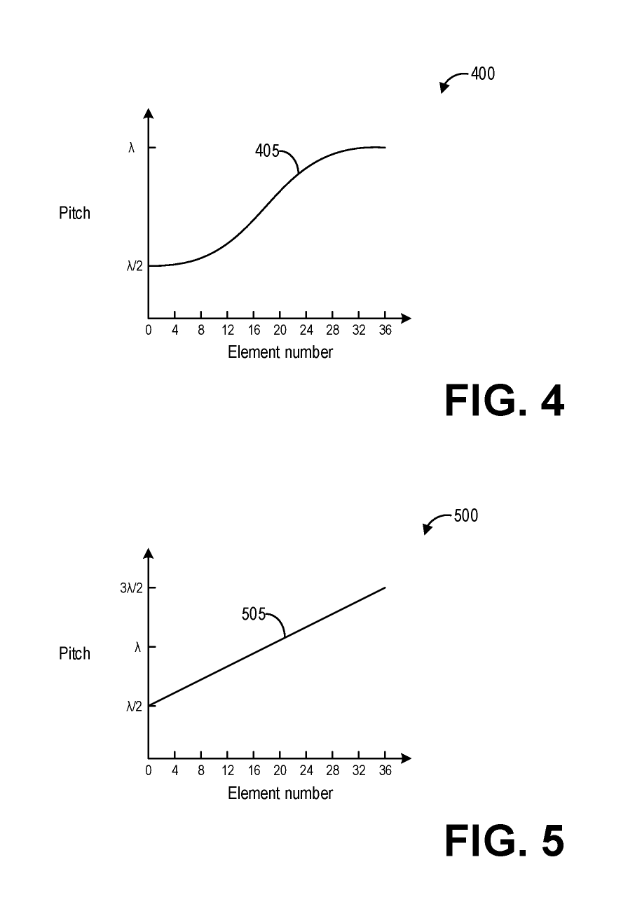

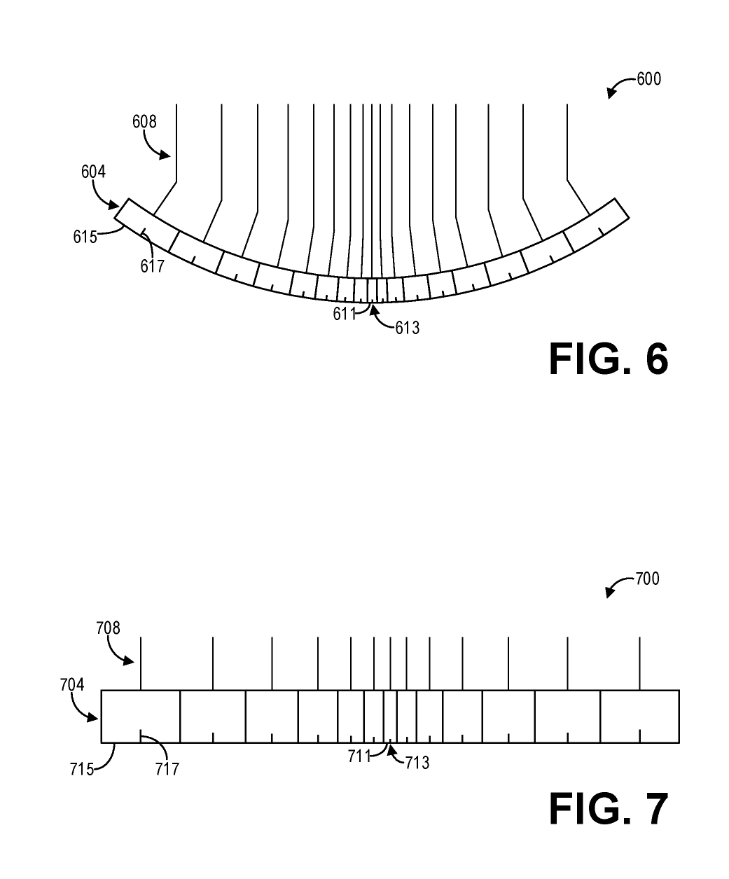

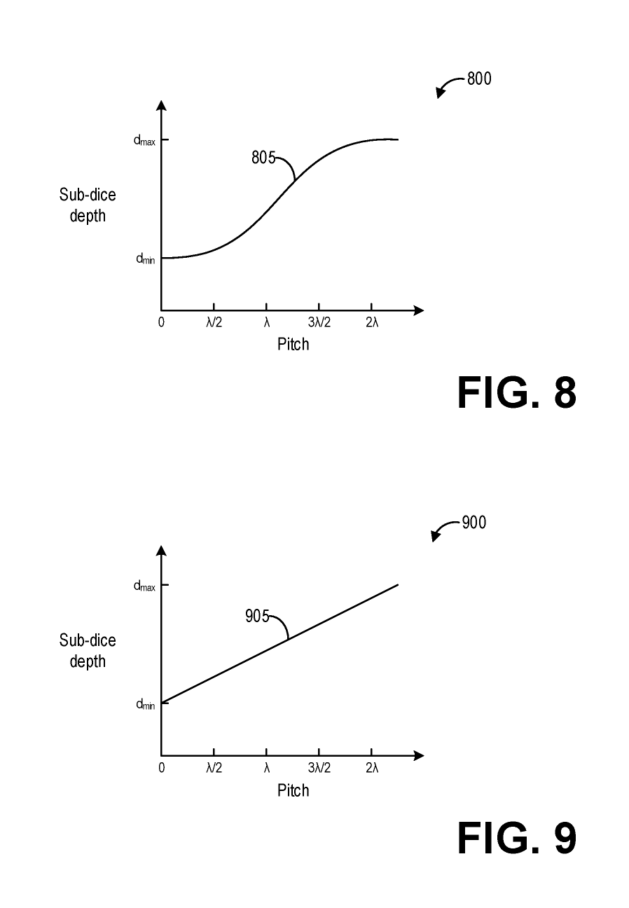

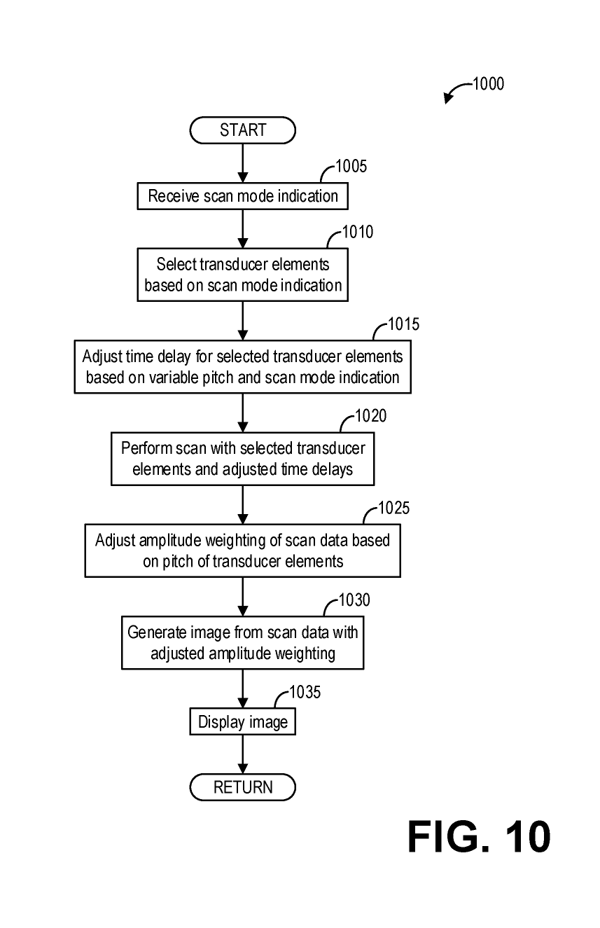

The following description relates to various embodiments of ultrasound transducers with variable pitch. In particular, systems and methods are provided for varying the pitch gradually across transducer elements. A transducer with variable pitch may be included in an ultrasound imaging system such as the system shown in FIG. 1. The transducer may comprise a curved array of transducer elements, wherein the pitch increases from fine pitch in a center of the array to a coarse pitch at an edge of the array, as depicted in FIG. 2. By providing a fine pitch in the middle of the array, elements in the central region of the transducer array which are approximately linear may be controlled in a phased array scan, while the full array of transducer elements may be otherwise used for an aperture translation scan. The variable pitch may be implemented in a linear transducer, such as the transducer shown in FIG. 3. The pitch varies across a range of pitches across all of the transducer elements, as shown in FIGS. 4 and 5. To improve the acoustic performance of the transducer array, the transducer elements may include sub-dice cuts in the face of each element, as shown in FIGS. 6 and 7. The depth of a dicing cut in a transducer element may depend on the pitch of the element, as shown in FIGS. 8 and 9, and thus the depth may also depend on the distance of the element from the center of the array if the pitch varies based on the distance. A method for operating a transducer probe with a variable pitch transducer array may include adjusting the transmission of ultrasonic waves to account for the variable pitch, as shown in FIG. 10. The method may further include adjusting scan data acquired during a scan to account for the variable pitch.

FIG. 1 is a schematic diagram of an ultrasound imaging system 100 in accordance with an embodiment of the invention. The ultrasound imaging system 100 includes a transmit beamformer 101 and a transmitter 102 that drive elements 104 within a transducer array, or probe, 106 to emit pulsed ultrasonic signals into a body (not shown). According to an embodiment, the transducer array 106 may be a one-dimensional transducer array probe. However, in some embodiments, the transducer array 106 may be a two-dimensional matrix transducer array probe. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit and/or the receive beamforming. For example, all or part of the transmit beamformer 101, the transmitter 102, the receiver 108, and the receive beamformer 110 may be situated within the probe 106.

The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including, to control the input of patient data, to change a scanning or display parameter, and the like. The user interface 115 may include one or more of the following: a rotary, a mouse, a keyboard, a trackball, hard keys linked to specific actions, soft keys that may be configured to control different functions, and a graphical user interface displayed on the display device 118.

The ultrasound imaging system 100 also includes a processor 116 to control the transmit beamformer 101, the transmitter 102, the receiver 108, and the receive beamformer 110. The processor 116 is in electronic communication with the probe 106. For purposes of this disclosure, the term “electronic communication” may be defined to include both wired and wireless communications. The processor 116 may control the probe 106 to acquire data. The processor 116 controls which of the elements 104 are active and the shape of a beam emitted from the probe 106. The processor 116 is also in electronic communication with a display device 118, and the processor 116 may process the data into images for display on the display device 118. The processor 116 may include a central processor (CPU) according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment the demodulation can be carried out earlier in the processing chain. The processor 116 is adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For example, an embodiment may acquire images at a real-time rate of 7-20 volumes/sec. The ultrasound imaging system 100 may acquire 2D data of one or more planes at a significantly faster rate. However, it should be understood that the real-time volume-rate may be dependent on the length of time that it takes to acquire each volume of data for display. Accordingly, when acquiring a relatively large volume of data, the real-time volume-rate may be slower. Thus, some embodiments may have real-time volume-rates that are considerably faster than 20 volumes/sec while other embodiments may have real-time volume-rates slower than 7 volumes/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, 10 Hz to 30 Hz. Images generated from the data may be refreshed at a similar frame-rate. Other embodiments may acquire and display data at different rates. For example, some embodiments may acquire data at a volume-rate of less than 10 Hz or greater than 30 Hz depending on the size of the volume and the intended application. A memory 120 is included for storing processed volumes of acquired data. In an exemplary embodiment, the memory 120 is of sufficient capacity to store at least several seconds' worth of volumes of ultrasound data. The volumes of data are stored in a manner to facilitate retrieval thereof according to its order or time of acquisition. The memory 120 may comprise any known data storage medium.

Optionally, embodiments of the present invention may be implemented using contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and combinations thereof, and the like. The image lines and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may be provided that reads the image volumes from a memory and displays an image in real time while a procedure is being carried out on a patient. A video processor module may store the images in an image memory, from which the images are read and displayed.

FIG. 2 shows a diagram illustrating an example curved transducer 200 with variable pitch according to an embodiment of the invention. Transducer 200 includes a plurality of transducer elements 204. The plurality of transducer elements 204 are arranged in an array 205, thereby forming a front face 207 from which ultrasonic waves (not shown) of wavelength λ may be emitted. As depicted, the array 205 may comprise a convex array 205, though it should be appreciated that in some examples the array may comprise a concave or a linear array. Each element of the plurality of transducer elements 204 is coupled to a corresponding control channel 208 through which signals (e.g., transmit signals, receive signals) may be communicated.

The inter-element spacing or pitch between adjacent transducer elements increases from a first pitch in a center of the array to a second pitch at an edge of the array. For example, neighboring transducer elements 211 and 212 are positioned in the center of the array with a first pitch 213, while neighboring transducer elements 215 and 216 are positioned at the edge of the array with a second pitch 217, where the second pitch 217 is greater than the first pitch 213. As depicted, the pitch between adjacent elements variably and gradually increases towards the edge of the array, such that the pitch of adjacent transducer elements between the elements 211 and 216 varies between the first pitch 213 and the second pitch 217.

Furthermore, the variable pitch may be symmetric about the center of the transducer array, as depicted, though it should be appreciated that in some examples the variable pitch may be asymmetric about the center of the array.

The transducer 200 may comprise a curved transducer such that the transducer 200 may be used for, as illustrative and non-limiting examples, both cardiac and abdominal imaging. For example, the transducer 200 may be used as a phased array for cardiac imaging by using only the center portion of the transducer 200. When used for abdominal imaging, it is desirable to use a larger active aperture.

The plurality of transducer elements 204 includes a central region 220 and lateral regions 221 and 223. In some examples, the transducer 200 is operable to scan in a first mode using transducer elements in the central region 220 as well as a second mode using transducer elements in the central region 220 and the lateral regions 221 and 223.

Although the transducer 200 is depicted with seventeen transducer elements 204, it should be understood that the transducer 200 may include a number of transducer elements greater than or less than seventeen. Furthermore, the transducer 200 may include an even number of transducer elements, such that there is no single central element such as element 211. For example, typical transducers may include 64 or 128 transducer elements.

As another illustrative example of a transducer with variable pitch, FIG. 3 shows a diagram illustrating an example linear transducer 300 with variable pitch according to an embodiment of the invention. Transducer 300 includes a plurality of transducer elements 304 arranged in a linear array 305, thereby forming a front face 307 of the transducer 300 from which ultrasonic waves (not shown) may be emitted. Each of the plurality of transducer elements 304 is coupled to a corresponding control channel 308 to enable communication of transmit signals and receive signals with a processor.

The central element 311 and the neighboring element 312 have a first pitch 321. In some examples, the first pitch 321 may be equal to half the wavelength λ of the ultrasonic waves emitted by the elements. In other examples, the first pitch 321 may be less than or greater than half the wavelength λ; for example, the first pitch 321 may be equal to the wavelength λ.

Elements positioned further away from the central element 311 include an increased pitch. For example, the element 312 and the neighboring element 313 have a second pitch 322 which is larger than the first pitch 321; the element 313 and the neighboring element 314 have a third pitch 323 which is larger than the second pitch 322; the element 314 and the neighboring element 315 have a fourth pitch 324 which is larger than the third pitch 323; the element 315 and the neighboring element 316 have a fifth pitch 325 which is larger than the fourth pitch 324; and the element 316 and the neighboring element 317 have a sixth pitch 326 which is larger than the fifth pitch 325. In examples wherein the first pitch 321 is equal to half the wavelength λ, the sixth pitch 326 may be equal to the wavelength λ, while the pitches 322, 323, 324, and 325 may range between half the wavelength λ and the wavelength λ. In other examples, the sixth pitch 326 may be 1.5 times the wavelength λ, such that the pitches 322, 323, 324, and 325 range between 0.5λ and 1.5λ.

It should be appreciated that the transducer 300 is illustrative and non-limiting. For example, while transducer 300 includes 13 elements, in some examples a linear transducer with variable pitch may include more or less than 13 elements. As non-limiting examples, a transducer array may include 64 or 128 transducer elements. Furthermore, while the transducer 300 depicts the variable pitch as symmetric about the center of the transducer array, it should be understood that in some examples, the variable pitch may be asymmetric about the center of the transducer array. As an illustrative and non-limiting example, the variable pitch may increase in one direction from λ/2 at the center of the transducer array to λ at a first edge of the transducer array, and the variable pitch may increase in the opposite direction from λ/2 at the center of the transducer array to 3λ/2 at a second edge of the transducer array.

FIG. 4 shows a graph 400 illustrating an example relation between pitch and transducer element position according to an embodiment of the invention. Specifically, graph 400 shows a plot 405 of inter-element spacing or pitch as a function of element number, where the element number denotes the position of a transducer element with respect to the center of the transducer array. For example, element number 0 may correspond to a central element, while element number 36 corresponds to an element at the edge of the transducer array (i.e., the element positioned furthest from the central element). It should be appreciated that the number of elements as well as the pitch range is illustrative and non-limiting.

As depicted, plot 405 increases monotonically from half the wavelength λ to the wavelength λ. The pitch may increase slowly for elements closer to the center or the edge, and may increase more rapidly for elements therebetween.

FIG. 5 shows a graph 500 illustrating another example relation between pitch and transducer element position according to an embodiment of the invention. Specifically, graph 500 shows a plot 505 of pitch as a function of element number. As depicted, plot 505 increases linearly from 0.5λ to 1.5λ. In such an example, the pitch increases by a constant amount between elements towards the edge of the array.

Thus, an ultrasound probe is provided, the ultrasound probe comprising a plurality of transducer elements arranged in an array, wherein a pitch between adjacent transducer elements increases from a first pitch in a center of the array to a second pitch at an edge of the array, and wherein the pitch gradually varies between the first pitch and second pitch for transducer elements positioned between the center and the edge. In this way, a single transducer may be operated in multiple modes, including a phased array mode and an aperture translation mode.

FIG. 6 shows a diagram illustrating an example curved transducer 600 with variable pitch and variable sub-dicing according to an embodiment of the invention. The transducer 600 may be substantially similar to the transducer 200 described herein above with regard to FIG. 2. For example, the transducer 600 includes a plurality of transducer elements 604 arranged in a convex array, wherein each element of the plurality of elements 604 is coupled to one control channel of a plurality of control channels 608.

Further, a small dicing cut or sub-dice may be made in the face of one or more transducer elements to improve the acoustic performance of the transducer elements 604. For example, as depicted, each element of the plurality of transducer elements 604 may include a sub-dice in the face of the element, though it should be appreciated that in some examples, one or more elements of the plurality of transducer elements 604 may not include a sub-dice.

In some examples, the depth of the sub-dice in a particular transducer element may be based on the pitch of the element or, similarly, the distance of the element from the center of the transducer array. For example, the central element 611 may include a sub-dice 613 with a minimum sub-dice depth, while the edge element 615 may include a sub-dice 617 with a maximum sub-dice depth. The transducer elements between the central element 611 and the edge element 615 may include sub-dices with depths that are greater than the minimum sub-dice depth and less than the maximum sub-dice depth. Further, the sub-dice depth of transducer elements between the central element 611 and the edge element 615 may increase towards the edge element 615.

In some examples, the minimum sub-dice depth may be zero, such that the central element 611 does not have a sub-dice. The maximum sub-dice depth may comprise the deepest cut allowable without negatively affecting the performance of the transducer element. As an illustrative example, the maximum sub-dice depth may be 10 micrometers to 300 micrometers, though it should be appreciated that the maximum sub-dice depth may depend on the size and/or the material composition of the transducer element.

It should be noted that each transducer element is coupled to a single control channel despite the presence of a sub-dice in the face of the transducer element. That is, a transducer element with a sub-dice is still treated as a single element, rather than two distinct elements.

As another example, FIG. 7 shows a diagram illustrating an example linear transducer 700 with variable pitch and variable sub-dicing according to an embodiment of the invention. The transducer 700 may be substantially similar to the transducer 300 described herein above with regard to FIG. 3. For example, the transducer 700 includes a plurality of transducer elements 704 arranged in a linear array, wherein each transducer element of the plurality of transducer elements 704 is coupled to a corresponding control channel of a plurality of control channels 708.

One or more of the transducer elements 704 may include a small dicing cut or sub-dice in the face of the one or more transducer elements. Similar to the transducer 600, the depth of the sub-dices in the transducer 700 may be based on the pitch of the element. For example, the central element 711 includes a sub-dice 713 with a first depth, while the edge element 715 includes a sub-dice 717 with a second depth, wherein the second depth is deeper than the first depth. Further, the elements between the central element 711 and the edge element 715 may include sub-dices with depths varying between the first depth and the second depth.

FIG. 8 shows a graph 800 illustrating an example relation between sub-slice depth and pitch according to an embodiment of the invention. Specifically, graph 800 shows a plot 805 of sub-dice depth as a function of pitch. Plot 805 comprises a sigmoidal curve such that the sub-dice depth increases monotonically as the pitch increases. The sub-dice depth increases from a minimum sub-dice depth dₘᵢₙ to a maximum sub-dice depth dₘₐₓ as the pitch increases.

The particular relation depicted in graph 800 is exemplary and non-limiting. For example, although plot 805 shows the minimum sub-dice depth dₘᵢₙ corresponding to a pitch of 0, in some examples the minimum sub-dice depth may correspond to a pitch of λ/2. Similarly, although plot 805 shows the maximum sub-dice depth dₘₐₓ corresponding to a pitch of 2λ, in some examples the maximum sub-dice depth may correspond to a pitch of λ. That is, the sub-dice depth may increase monotonically from dₘᵢₙ to dₘₐₓ as the pitch increases from λ/2 to λ.

Thus, the depth of a sub-dice in a transducer element may correspond to the pitch of the transducer element. Since the pitch of a transducer element depends on the distance of the transducer element from the center of the transducer array, as described herein, it should be appreciated that the depth of a sub-dice in a transducer element depends on the distance of the transducer element from the center of the transducer array.

FIG. 9 shows a graph 900 illustrating another example relation between sub-slice depth and pitch according to an embodiment of the invention. Specifically, graph 900 shows a plot 905 of sub-dice depth as a function of pitch. Plot 905 increases linearly from a minimum sub-dice depth dₘᵢₙ to a maximum sub-dice depth dₘₐₓ. Similar to the plot 805 described herein above, the plot 905 is illustrative and non-limiting. Although plot 905 depicts the sub-dice depth increasing linearly from the minimum sub-dice depth dₘᵢₙ to the maximum sub-dice depth dₘₐₓ as the pitch increases from 0 to 2λ, in some examples the sub-dice depth may increase linearly from dₘᵢₙ to dₘₐₓ as the pitch increases from λ/2 to λ.

FIG. 10 shows a high-level flow chart illustrating an example method 1000 for performing an ultrasound scan according to an embodiment of the invention. Method 1000 will be described herein with reference to the system depicted in FIGS. 1 and 2, though it should be understood that the method may be applied to other systems and components without departing from the scope of this disclosure. Method 1000 may be carried out by processor 116, and may be stored as executable instructions in non-transitory memory of the processor 116.

Method 1000 begins at 1005. At 1005, method 1000 includes receiving a scan mode indication. The scan mode indication comprises a selection of the scan mode by the operator of the ultrasound system. To that end, the scan mode indication may be received, for example, via a user interface such as user interface 115. Scan modes may include, but are not limited to, a phased array mode and a aperture translation mode. In the phased array mode, transducer elements in the central region of the transducer array may be controlled to transmit a steered ultrasound beam into the subject. The active aperture, or the subset of transducer elements actively emitting ultrasound waves, remains static in the phased array mode. In the aperture translation mode, transducer elements in the central region and the lateral regions of the transducer array may be controlled to transmit an ultrasound beam into the subject. Further, in the aperture translation mode, the active aperture may be translated across the physical aperture of the ultrasound probe.

After receiving the scan mode indication, method 1000 continues to 1010. At 1010, method 1000 includes selecting transducer elements based on the scan mode indication. Specifically, the method selects transducer elements to form the aperture of the ultrasound probe. For example, if the phased array mode is selected at 1005, the method selects transducer elements in the central region of the transducer array. The selected transducer elements in the central region of the transducer array thus form the active aperture of the ultrasound probe during a phased array scan. As another example, if the aperture translation mode is selected at 1005, the method selects transducer elements across the transducer array, including elements in the central region and the lateral regions of the transducer array. The selected elements comprise a physical aperture of the ultrasound probe, while the active aperture (i.e., the elements emitting ultrasonic waves at a given time) comprises a subset of the physical aperture and is translated across the physical aperture during a scan with the aperture translation mode.

At 1015, method 1000 includes adjusting a time delay for the selected transducer elements based on the variable pitch and the scan mode indication. Specifically, the time delay applied to a transmit signal for a given element of the selected transducer elements is adjusted to account for the distance of the given element from the center of the transducer array, such that, as a non-limiting example, the ultrasonic wave generated by the given element arrives at the desired location simultaneously with ultrasonic waves generated by other transducer elements. Since the pitch between elements increases gradually from the center to the edge of the transducer array, the time delays will be longer or shorter, depending on the scan mode and the position of the element, in comparison to the time delays that would be applied to transmit signals for a transducer array with constant pitch.

Continuing at 1020, method 1000 includes performing a scan with the selected transducer elements and the adjusted time delays. Performing the scan includes applying transmit signals to each of the selected transducer elements with the adjusted time delays such that the selected transducer elements generate ultrasonic waves into a body. Performing the scan further includes receiving, with the transducer elements, echoes of the ultrasonic waves after the waves reflect off structures within the body.

At 1025, method 1000 includes adjusting the amplitude weighting of scan data based on the pitch of the transducer elements. Specifically, since the pitch increases from fine to coarse for elements at the center of the array to elements at the edge of the array, the scan data received at the center of the array is automatically emphasized in comparison to the scan data received at the edge of the array. The amplitude weighting may thus be adjusted to counter this effect, for example by reducing the amplitude weighting for scan data received by central elements in comparison to the amplitude weighting for scan data received by edge elements.

Continuing at 1030, method 1000 includes generating an image from the scan data with the adjusted amplitude weighting. At 1035, method 1000 includes displaying, via a display device such as display device 118, the image. Method 1000 then ends.

Thus, a method is provided that comprises: transmitting, with an ultrasound transducer comprising a plurality of transducer elements arranged in an array, acoustic waves into a subject, wherein a pitch of each transducer element of the plurality of transducer elements variably increases from a first pitch at a center of the array to a second pitch at an edge of the array; receiving, with the ultrasound transducer, echoes of the transmitted acoustic waves; and generating, with a processor communicatively coupled to the ultrasound transducer, an image from the received echoes after adjusting the received echoes based on the pitch.

A technical effect of the disclosure is the adjustment of time delays of transmit signals applied to transducer elements, wherein the adjustment of a time delay for a particular transducer element is based on the pitch of the transducer element. Another technical effect of the disclosure is the adjustment of amplitude weights for scan data based on the pitch of the transducer elements that receive the scan data.

In one embodiment, an ultrasound probe comprises a plurality of transducer elements arranged in an array, wherein a pitch between adjacent transducer elements increases from a first pitch in a center of the array to a second pitch at an edge of the array, and wherein the pitch gradually varies between the first pitch and second pitch for transducer elements positioned between the center and the edge.

In a first example of the ultrasound probe, each of the plurality of transducer elements is operable to emit an ultrasonic wave with a wavelength, and the first pitch is half the wavelength and the second pitch is greater than or equal to the wavelength. In a second example of the ultrasound probe optionally including the first example, one or more transducer elements of the plurality of transducer elements includes a sub-dice in the one or more transducer elements, wherein a depth of the sub-dice in the one or more transducer elements is based on a pitch of the one or more transducer elements. In a third example of the ultrasound probe optionally including one or more of the first and second examples, the depth of the sub-dice increases monotonically as a function of distance from the center. In a fourth example of the ultrasound probe optionally including one or more of the first through third examples, the depth of the sub-dice increases linearly as a function of the distance from the center. In a fifth example of the ultrasound probe optionally including one or more of the first through fourth examples, the pitch between adjacent transducer elements increases monotonically from the center to the edge as a function of distance from the center. In a sixth example of the ultrasound probe optionally including one or more of the first through fifth examples, the pitch between adjacent transducer elements increases linearly from the center to the edge as a function of distance from the center. In a seventh example of the ultrasound probe optionally including one or more of the first through sixth examples, the plurality of elements are arranged in a convex array. In an eighth example of the ultrasound probe optionally including one or more of the first through seventh examples, the plurality of transducer elements includes a central region and lateral regions, wherein the ultrasound probe is operable to scan in a first mode using transducer elements in the central region and is further operable to scan in a second mode using transducer elements in the central region and the lateral regions.

In another embodiment, a system comprises: a transducer including a plurality of elements arranged in an array, wherein a pitch between adjacent elements increases from a first pitch in a center of the array to a second pitch at an edge of the array, wherein the pitch varies between the first pitch and the second pitch for elements between the center and the edge of the array; a transmitter coupled to the transducer and configured to apply a separate signal pulse to each element of the plurality of elements; a receiver coupled to the transducer and configured to receive an echo signal produced by each element; and a processor coupled to the receiver and configured with instructions stored in non-transitory memory that when executed cause the processor to generate an image using echo signals received by one or more elements of the plurality of elements during a scan.

In a first example of the system, the processor is further configured with instructions stored in the non-transitory memory that when executed cause the processor to adjust a delay of the separate signal pulse applied to each element based on the pitch of each element. In a second example of the system optionally including the first example, the processor is further configured with instructions stored in the non-transitory memory that when executed cause the processor to adjust an amplitude weighting applied to the echo signal produced by each element based on the pitch of each element. In a third example of the system optionally including one or more of the first and second examples, the amplitude weighting is reduced for echo signals produced by elements in the center of the array compared to the amplitude weighting for echo signals produced by elements at the edge of the array. In a fourth example of the system optionally including one or more of the first through third examples, one or more elements of the plurality of elements includes a sub-dice with a depth based on a pitch of the one or more elements. In a fifth example of the system optionally including one or more of the first through fourth examples, the transducer includes a central region and two lateral regions, wherein the transmitter applies the separate signal pulses to elements in the central region in a first mode, and wherein the transmitter applies the separate signal pulses to elements in the central region and the two lateral regions in a second mode.

In yet another embodiment, a method comprises: transmitting, with an ultrasound transducer comprising a plurality of transducer elements arranged in an array, acoustic waves into a subject, wherein a pitch of each transducer element of the plurality of transducer elements variably increases from a first pitch at a center of the array to a second pitch at an edge of the array; receiving, with the ultrasound transducer, echoes of the transmitted acoustic waves; and generating, with a processor communicatively coupled to the ultrasound transducer, an image from the received echoes after adjusting the received echoes based on the pitch.

In a first example of the method, the method further comprises receiving, via a user interface communicatively coupled to the processor, a selection of a scan mode, and selecting, based on the selection, at least a portion of transducer elements of the plurality of transducer elements for transmitting the acoustic waves into the subject. In a second example of the method optionally including the first example, the method further comprises adjusting a time delay of a transmit signal applied to one or more transducer elements of the plurality of transducer elements based on a pitch of the one or more transducer elements, wherein the ultrasound transducer transmits the acoustic waves into the subject responsive to the transmit signal. In a third example of the method optionally including one or more of the first and second examples, adjusting the received echoes based on the pitch comprises adjusting amplitude weighting of one or more echoes of the received echoes based on a pitch of a transducer element that receives the one or more echoes of the received echoes. In a fourth example of the method optionally including one or more of the first through third examples, the method further comprises displaying, via a display device communicatively coupled to the processor, the generated image.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An ultrasound probe, comprising:
a plurality of transducer elements arranged in an array, wherein a pitch between adjacent transducer elements increases from a first pitch in a center of the array to a second pitch at an edge of the array, and wherein the pitch gradually varies between the first pitch and second pitch for transducer elements positioned between the center and the edge.

2. The ultrasound probe of claim 1, wherein each of the plurality of transducer elements is operable to emit an ultrasonic wave with a wavelength, and wherein the first pitch is half the wavelength and the second pitch is greater than or equal to the wavelength.

3. The ultrasound probe of claim 1, wherein one or more transducer elements of the plurality of transducer elements includes a sub-dice in the one or more transducer elements, wherein a depth of the sub-dice in the one or more transducer elements is based on a pitch of the one or more transducer elements.

4. The ultrasound probe of claim 3, wherein the depth of the sub-dice increases monotonically as a function of distance from the center.

5. The ultrasound probe of claim 4, wherein the depth of the sub-dice increases linearly as a function of the distance from the center.

6. The ultrasound probe of claim 1, wherein the pitch between adjacent transducer elements increases monotonically from the center to the edge as a function of distance from the center.

7. The ultrasound probe of claim 6, wherein the pitch between adjacent transducer elements increases linearly from the center to the edge as a function of distance from the center.

8. The ultrasound probe of claim 1, wherein the plurality of elements are arranged in a convex array.

9. The ultrasound probe of claim 1, wherein the plurality of transducer elements include a central region and lateral regions, wherein the ultrasound probe is operable to scan in a first mode using transducer elements in the central region and is further operable to scan in a second mode using transducer elements in the central region and the lateral regions.

10. A system, comprising:
a transducer including a plurality of elements arranged in an array, wherein a pitch between adjacent elements increases from a first pitch in a center of the array to a second pitch at an edge of the array, wherein the pitch varies between the first pitch and the second pitch for elements between the center and the edge of the array;
a transmitter coupled to the transducer and configured to apply a separate signal pulse to each element of the plurality of elements;
a receiver coupled to the transducer and configured to receive an echo signal produced by each element;
a processor coupled to the receiver and configured with instructions stored in non-transitory memory that when executed cause the processor to generate an image using echo signals received by one or more elements of the plurality of elements during a scan.

11. The system of claim 10, wherein the processor is further configured with instructions stored in the non-transitory memory that when executed cause the processor to adjust a delay of the separate signal pulse applied to each element based on the pitch of each element.

12. The system of claim 10, wherein the processor is further configured with instructions stored in the non-transitory memory that when executed cause the processor to adjust an amplitude weighting applied to the echo signal produced by each element based on the pitch of each element.

13. The system of claim 12, wherein the amplitude weighting is reduced for echo signals produced by elements in the center of the array compared to the amplitude weighting for echo signals produced by elements at the edge of the array.

14. The system of claim 10, wherein one or more elements of the plurality of elements includes a sub-dice with a depth based on a pitch of the one or more elements.

15. The system of claim 10, wherein the transducer includes a central region and two lateral regions, wherein the transmitter applies the separate signal pulses to elements in the central region in a first mode, and wherein the transmitter applies the separate signal pulses to elements in the central region and the two lateral regions in a second mode.

16. A method, comprising:
transmitting, with an ultrasound transducer comprising a plurality of transducer elements arranged in an array, acoustic waves into a subject, wherein a pitch of each transducer element of the plurality of transducer elements variably increases from a first pitch at a center of the array to a second pitch at an edge of the array;
receiving, with the ultrasound transducer, echoes of the transmitted acoustic waves; and
generating, with a processor communicatively coupled to the ultrasound transducer, an image from the received echoes after adjusting the received echoes based on the pitch.

17. The method of claim 16, further comprising receiving, via a user interface communicatively coupled to the processor, a selection of a scan mode, and selecting, based on the selection, at least a portion of transducer elements of the plurality of transducer elements for transmitting the acoustic waves into the subject.

18. The method of claim 16, further comprising adjusting a time delay of a transmit signal applied to one or more transducer elements of the plurality of transducer elements based on a pitch of the one or more transducer elements, wherein the ultrasound transducer transmits the acoustic waves into the subject responsive to the transmit signal.

19. The method of claim 16, wherein adjusting the received echoes based on the pitch comprises adjusting amplitude weighting of one or more echoes of the received echoes based on a pitch of a transducer element that receives the one or more echoes of the received echoes.

20. The method of claim 16, further comprising displaying, via a display device communicatively coupled to the processor, the generated image.
